(19) United States
(12) Patent Application Publication
(10) Pub. No.: US 2003/0186888 A1
(43) Pub. Date: Oct. 2, 2003

(54) Diastereo analog of peptide SPFK-amide with selective anti-microbial activity and a method thereof

(76) Inventors: Chilukuri Subbalakshmi, Hyderabad (IN); Ramakrishnan Nagaraj, Hyderabad (IN); Narasimhaiah Sitaram, Hyderabad (IN)

Correspondence Address: John P. White, Cooper & Dunham LLP, 1185 Avenue of the Americas, New York, NY, 10036, US

(21) Appl. No.: 10/113,512

(22) Filed: Mar. 28, 2002

(51) Int. Cl. A61K 38/10; C07K 7/08; C12P 21/06

(52) U.S. Cl. 514/14; 435/69.1; 530/327

(57) ABSTRACT

The present invention relates to a novel Diastereo analog Dsam of peptide SPFK-amide of amino acid sequence PKLLKTFLSKWIG with D-Leu residues at positions 4 and 8 of analog, having selective anti-microbial activity and no hemolytic activity of said SPFK, and a method of producing said Diastereo analog.

### FIELD OF THE INVENTION

The present invention relates to a novel Diastereo analog Dsam of peptide SPFK-amide with D-Leu residues at positions 4 and 8 of analog, having selective antimicrobial activity and a method thereof.

### BACKGROUND AND PRIOR ART WORK

Antimicrobial peptides have now been recognized as part of innate immune system in organisms throughout the evolutionary scale (1-10). They kill microorganisms rapidly by permeabilizing their membranes. (1, 11). The mechanism of action of these peptides is thus different from therapeutically used antibiotics.

In recent years, infection caused by microbes resistant to clinically used antibiotics has resulted in a serious threat to public health, globally (12-16). To treat infections caused by multidrug resistant microbes, search for radically new structural class of antibiotics has not only become inevitable but urgent. Many endogenous antimicrobial peptides which are generally localized within cells like defensins exhibit both antimicrobial as well as cytolytic activity against normal mammalian cells (2). However, peptides found in body fluids and mucosal surfaces like cecropins, magainins and seminalplasmin possess activity only against microbial cells with no or minimal activity against normal mammalian cells (17-22).

Host-defense peptides are generally composed of more than 20 amino acids and peptides like defensins have 3 disulfide bridges (1-10). These features do not favor their use as therapeutic agents. Thus, understanding the structural requirements in a peptide for specific and improved activity against microbial cells would be of considerable help in designing peptides that could be useful in therapies.

Short linear peptides, possessing specific antimicrobial activity, would be attractive candidates as therapeutic agents. A 13-residue synthetic peptide SPF, PKLLETFLSKWIG, which corresponds to the most hydrophobia segment of the 47-residue antimicrobial peptide bovine seminal plasmin exhibits both antimicrobial and hemolytic activity (23, 24).

Applicants have earlier shown that replacement of E by K in SPF (i.e. SPFK) results in enhanced antimicrobial activity without a concomitant change in its hemolytic activity (24). In an effort to decrease or abolish the undesirable hemolytic activity in SPFK and also to gain further insights into structure-activity relationships in short peptides, Applicants have examined the activities of retro and diastereo analogs of SPFK.

Antimicrobial peptides, from a variety of sources, like cecropins, magainins, dermaseptin and seminalplasmin are known to exhibit activity specifically against microorganisms with minimal hemolytic activity (1-11). Microbial cell membranes, specifically the lipopolysaccharide and lipid components, are the targets of action for these peptides (1, 11). There are several other peptides like melittin and pardaxin which are active on both microbial and mammalian cells (11, 33-35). Peptides like δ-toxin exhibit only hemolytic and show no antimicrobial activity (36). Knowledge of structural determinants that dictate target specificity would be essential for designing peptides with specific and potent antimicrobial activity and these peptides could have potential therapeutic applications.

In the case of melittin, a synthetic retro peptide, in which the direction of the peptide backbone had been reversed, showed reduced hemolytic activity without change in its antimicrobial activity (37). In our present study, we have observed that both retro peptides with the C-terminus as acid or amide possessed antimicrobial activity similar to that of SPFK and also could lyse erythrocytes, though with marginally lower potency than the parent peptide. Thus, reversing the sequence of SPFK did not result in the loss of hemolytic activity. Both the retro peptides were also bactericidal and were capable of permeabilizing both bacterial OM and IM at their MICs and their mechanism of action appeared to be same as that of their parent peptide, SPFK (24).

The single Trp residue in melittin plays a major role in its hemolytic activity, as deletion of this residue or its replacement results in a large reduction of this activity (30,38). Based on molecular modeling studies it has been proposed that the Trp residue would be solvent exposed on folding where as in retro-melittin, it would be shielded from solvent (37). This difference in the positioning of the single Trp residues is presumed to be the reason for the differential activities of melittin and retro melittin. SPFK also has a single Trp residue and an analog of SPFK lacking its two aromatic residues retains its antimicrobial activity, but is non-hemolytic, suggesting an important role for these residues for its hemolytic activities (39). It is conceivable that in both SPFK and its retro analogs, the Trp residues remain exposed to solvent and hence a reduction of hemolytic activity is not observed on reversing the sequence.

It appears from several studies especially on pardaxin, melittin and dermaseptin that hydrophobicity, net positive charge and propensity to adopt a-helical structure in hydrophobia environment are essential prerequisites for a peptide to exhibit hemolytic activity (40-49). Disruption of helical structure in these peptides, especially in the amino terminal region, generally leads to specific loss of hemolytic activity without adversely affecting their antimicrobial activity (40-49). The strategies that have been employed to disrupt helical structures are introduction of either Pro residues, which is known to be a structure breaker (43), or D enantiomers of amino acid residues in the middle of the sequence (41, 44, 46, 47).

### OBJECTS OF THE INVENTION

The main object of the present invention is to develop an analog of SPFK with only anti-microbial activity.

Another object of the present invention is to develop an analog of SPFK with reduced or no hemolytic activity.

Yet another object of the present invention is to develop a method to produce diastereo analog of SPFK-amide.

Still another object of the present invention is to understand the mechanism involved with anti-microbial activty of diastereo analogs of SPFK-amide.

Still another object of the present invention is to understand the behaviour of analogs of SPFK-amide.

### SUMMARY OF THE PRESENT INVENTION

The present invention relates to a novel Diastereo analog Dsam of peptide SPFK-amide of amino acid sequence PKLLKTFLSKWIG with D-Leu residues at positions 4 and 8 of analog, having selective anti-microbial activity and no hemolytic activity of said SPFK, and a method of producing said Diastereo analog.

### DETAILED DESCRIPTION OF THE PRESENT INVENTION

Accordingly, the present invention relates to a novel Diastereo analog Dsam of peptide SPFK-amide of amino acid sequence PKLLKTFLSKWIG with D-Leu residues at positions 4 and 8 of analog, having selective anti-microbial activity and no hemolytic activity of said SPFK, and a method of producing said Diastereo analog.

In one embodiment of the present invention, a 13-residue diastereo analog Dsam of SEQ ID No. 1 of peptide SPFK-amide having amino acid D-leucine at positions 4 and 8.

In another embodiment of the present invention, wherein said analog shows hydrophobicity (H) value raging between 0.19-0.21.

In yet another embodiment of the present invention, wherein said analog shows hydrophobicity moment of about 0.043.

In still another embodiment of the present invention, wherein said analog shows net charge of about +4.

In still another embodiment of the present invention, wherein said analog shows retention time of about 36.62 minutes on Reverse Phase High-Performance Liquid Chromatography.

In still another embodiment of the present invention, wherein said analog forms unordered structure in both aqueous and trifluoroethanol mediums.

In still another embodiment of the present invention, wherein said analog shows lower propensity for helical structure due to presence of D-Leu residues.

In still another embodiment of the present invention, wherein said analog shows antimicrobial activity.

In still another embodiment of the present invention, wherein said analog shows reduced hemolytic activity as compared to SPFK.

In further embodiment of the present invention, a method of producing a 13-residue diastereo analog Dsam of SEQ ID No. 1 of peptide SPFK-amide having amino acid D-leucine at positions 4 and 8.

In another embodiment of the present invention, synthesizing said peptide by solid phase peptide synthesis method

In yet another embodiment of the present invention, purifying the said synthesized deprotected peptide by chromatography.

In still another embodiment of the present invention, confirming the purity of peptide by mass spectrometery.

In still another embodiment of the present invention, wherein said peptide is purified by High pressure Liquid Chromatography.

In still another embodiment of the present invention, wherein said peptide shows about same anti-microbial activity as SPFK.

In still another embodiment of the present invention, wherein said peptide shows reduced hemolytic activity as compared to SPFK.

In still another embodiment of the present invention, wherein said peptide shows antimicrobial activity by permeabilization of microbial membrane.

In still another embodiment of the present invention, wherein said analog shows antimicrobial activity at concentration 50 μg/ml and above.

In still another embodiment of the present invention, wherein said analog is administered through oral, nasal, intravenous, and/or intradermal route.

In further embodiment of the present invention, a composition useful for antimicrobial activity, said composition comprising diastereo analog Dsam of claim 1 and optionally pharmaceutically acceptable additives.

In another embodiment of the present invention, a composition as claimed in claim 17, wherein said analog and additives are in the ratio ranging between 1:1 to 1:10.

In yet another embodiment of the present invention, wherein said additives are selected from a group comprising cellulose, magnesium stearate, calcium carbonate, starch-gelatin paste, and/or pharmaceutically acceptable carriers, excipient, diluent, or solvent.

In still another embodiment of the present invention, wherein the additives show no adverse effect on the activity of the analog.

In still another embodiment of the present invention, wherein said analog shows antimicrobial activity at concentration 50 μg/ml and above.

In still another embodiment of the present invention, wherein said composition shows reduced hemolytic activity as compared to SPFK.

In still another embodiment of the present invention, wherein said composition is administered through oral, nasal, intravenous, and/or intradermal route.

In an embodiment of the present invention, for peptide synthesis and purification, sequences of the peptides used in this work are shown in Table 1. Synthesis and characterization of SPFK has been reported earlier (24).

The four analogs of SPFK reported in this Application i.e., retro SPFK acid (RSac), retro SPFK amide (RSam), diastereo SPFK acid (DSac) and diastereo SPFK amide (DSam) are synthesized using the Multipin Peptide Synthesis kit from Chiron Technologies.

Macrocrowns were used with Fmoc chemistry (25). The synthetic peptides were cleaved and deprotected using 95% trifluoroacetic acid (TFA) containing 5% of 1:3 mixture of ethanedithiol (EDT) and thioanisole (TA) for 16-18 h at room temperature. Cleaved peptides were purified on Hewlett Packard 1100 series high pressure liquid chromatography (HPLC) instrument using a (iBondapak Cis column (3.9×300 mm) (Waters).

The solvent system consisted of aqueous 0.1% TFA as mobile phase A and 0.1% TFA in acetonitrile as solvent B. A linear gradient from 15-60% B in A, with a flow rate of 1 ml/min was used, with peak detection at 280 nm. Purity of peptides were confirmed by matrix assisted laser desorption ionization time of flight mass spectrometry.

In further embodiment of the present invention, for determining antimicrobial activity, antimicrobial activity was assayed as follows: Different concentrations of peptides were added to 1 ml of synthetic medium [KH2PO4, 10.5 g; K2HPC>4, 4.5 g; (NH4)2SO4, 1 g; MgSO4, 0.1 g; L-arginine, 0.1 g and glucose 1% for one litre of medium] for E. coli or nutrient broth for other microorganisms, containing an inocula of the test organism adjusted to ˜10⁶ CFU/ml. Microbial growth was determined by monitoring the increase in ODeoo after incubating under aerobic conditions at 37° C. for 6-8 h (30° C. for P. aeruginosa). The bactericidal activity of the peptides was determined by plating suitably diluted cultures after incubation with the peptides on nutrient agar plates. The plates were incubated at 37° C. for 18 h for colony counting. Outer membrane permeabilization assays were done by fluorescent monitoring of uptake of N-phenyl-1-N-naphthyl amine (NPN) (26). E. co//W 160.37 cells were grown to late logarithmic phase in nutrient broth and cells obtained were washed twice with 5 mM HEPES buffer (pH 7.4). Aliquots of 1 ml of the cells so prepared and adjusted to an ODeoo of 0.1 in the same buffer containing 10 μM of NPN was taken for each experiment. The excitation monochromator was set at 350 nm and emission at 420 nm was continuously monitored after addition of the peptide from an aqueous stock solution. The experiment was carried out at 25° C. with continuous stirring on a Hitachi F4010 spectrofluorimeter. Inner membrane permeability of E. coli W 160.37 was monitored by measuring the p-galactosidase activity in preinduced cells (with 5×10′⁴ M IPTG), in the presence of different concentration of the peptide using 0-nitro phenyl-3-D-galactoside (ONPG)(27).

In further embodiment of the present invention, hemolytic activity of the peptides was assessed on freshly isolated rat erythrocytes. Heparinized rat blood in saline was initially centrifuged at 1000×g for 10 min to remove the buffy coat. The erythrocyte suspension was further washed thrice with 5 mM Hepes, pH 7.4 containing 150 mM NaCl. Aliquots of cell suspension (˜10⁶ cells) in eppendorf tubes were incubated with the peptide in duplicates at 37° C. for 30 min with gentle mixing. The tubes were then centrifuged and the absorbance of the supernatants was measured at 540 nm. The lysis obtained with 1% triton X 100 was taken as 100%.

In further embodiment of the present invention, CD spectra were recorded in 5 mM Hepes pH 7.4 and Trifluoroethanol (TFE) on a Jasco J-715 automatic recording spectropolarimeter at 25° C. using a quartz cell of 1 mm path length. Calibration was carried out with d-camphorsulfonic acid. Data are represented as mean residue ellipticties. Fractional helicities were calculated as fₕ={[θ]₂₂₂−[θ]⁰₂₂₂}/[θ]¹⁰⁰₂₂₂ from the experimentally obtained mean residue ellipticity at [9J222 nm and values for [θ] corresponding to 100% and 0% helical content at this wavelength. Values of [θ]¹⁰⁰₂₂₂=−28,400 deg cm² dmol′¹, [θ]°₂₂₂=−2000 deg cm² dmol⁻¹ were taken for calculations (28).

In further embodiment of the present invention, for Calmodulin interaction studies, the interaction with calmodulin (CaM) was studied by monitoring the Trp fluorescence of the peptides. A fixed concentration of peptide was titrated individually with bovine brain calmodulin (Sigma, St. Louis, USA) in 5 mM Tris-HCI buffer (pH 7.4) containing 1 mM CaCl2. The concentrations of the peptides used were 3 μM. The excitation wavelength was set at 295 nm and emission was monitored between 320 and 400 nm. When the saturation value of λ-max and fluorescence emission was reached, EDTA was added to a final concentration of 2 mM. All the spectra were corrected for dilutions and subtracted from suitable blanks. Fluorescence studies were carried out on Hitachi F4010 spectrofluorimeter at 25° C.

In further embodiment of the present invention, the sequences of the peptides synthesized are shown in Table 1 at the end. Retro SPFK acid (RSac) was generated by synthesizing the sequence of SPFK in the reverse order. The corresponding peptide with a C-terminal amide yielded retro SPFK amide (RSam). Diastereo SPFK (DSac) was synthesized by introducing D-Leu residues at positions 4 and 8 of SPFK. DSam is the corresponding C-terminal amide. SPFK has propensity for a-helical structure in membrane mimicking environment (24).

### BRIEF DESCRIPTION OF THE ACCOMPANYING DRAWINGS

### Legends to Figures

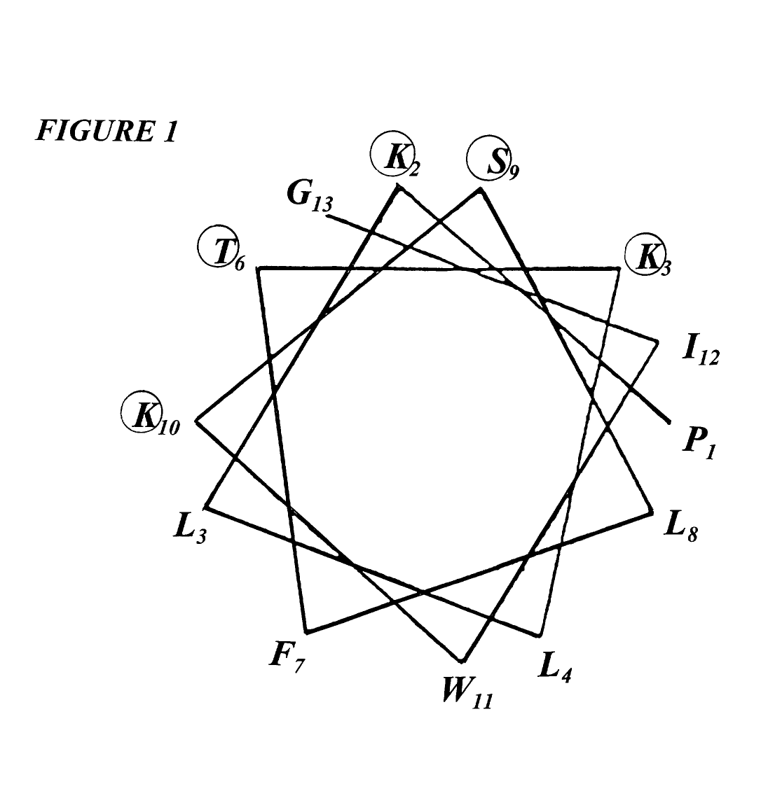

FIG. 1. Helical wheel projection of SPFK. Polar residues are circled.

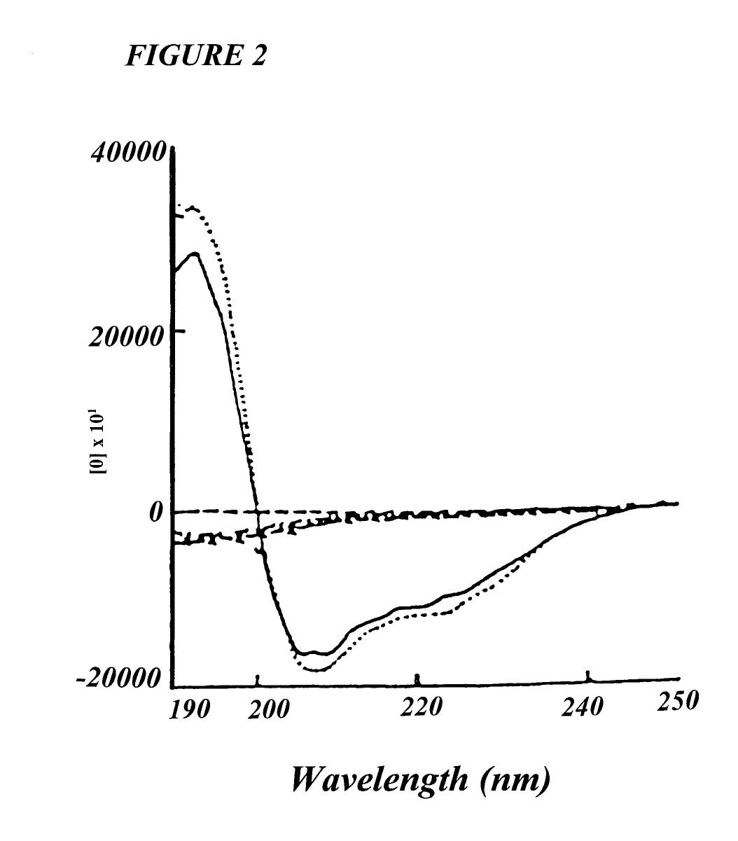

FIG. 2. CD spectra of SPFK analogs in TFE. Peptide concentrations were 0.05 mM. RSac(______ ), RSam (.....), DSac (-.-.-) and DSam (-x-x-).

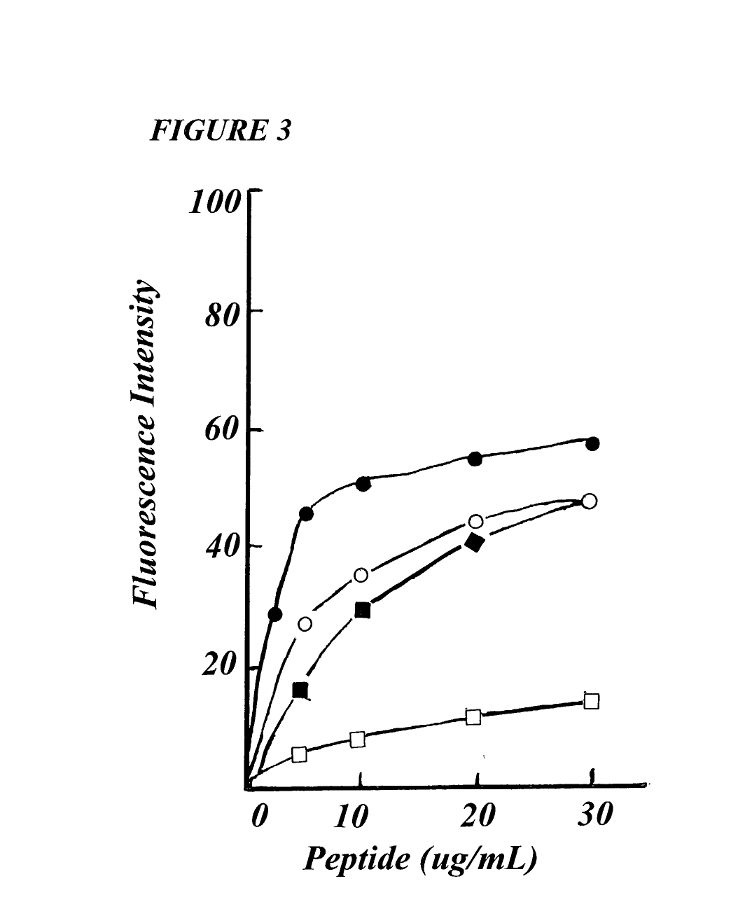

FIG. 3. Permeabilization of outer membrane(OM) of E. coli by SPFK analogs. Peptide-mediated OM-permeabilization of E. coli was measured as increase in the fluorescence intensity of N-phenyl-1-naphthylamine (NPN) in the presence of various concentrations of SPFK analogs, RSac (O), RSam (•), Dsac (D)and DSam(B).

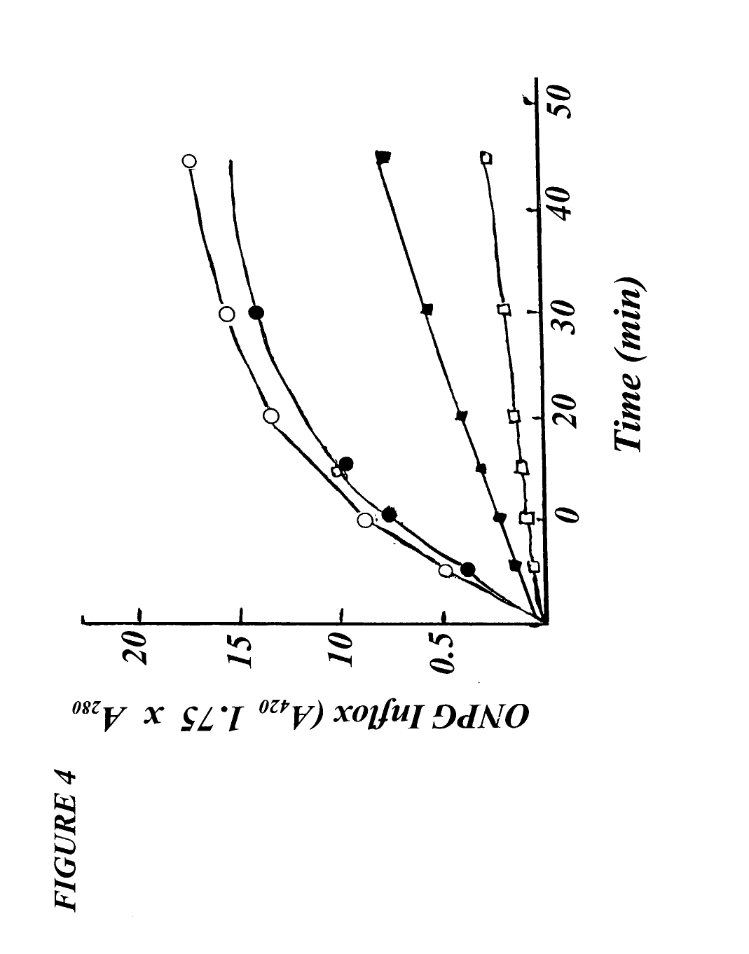

FIG. 4. Permeabilization of the inner membrane (IM) of E. coli by SPFK analogs. Peptide-mediated IM-permeabilization of E. coli was measured as increase in the influx of ONPG as a function of time in to the cells in the presence of 20 fig/ml of different analogs of SPFK. Symbols used are same as in FIG. 1.

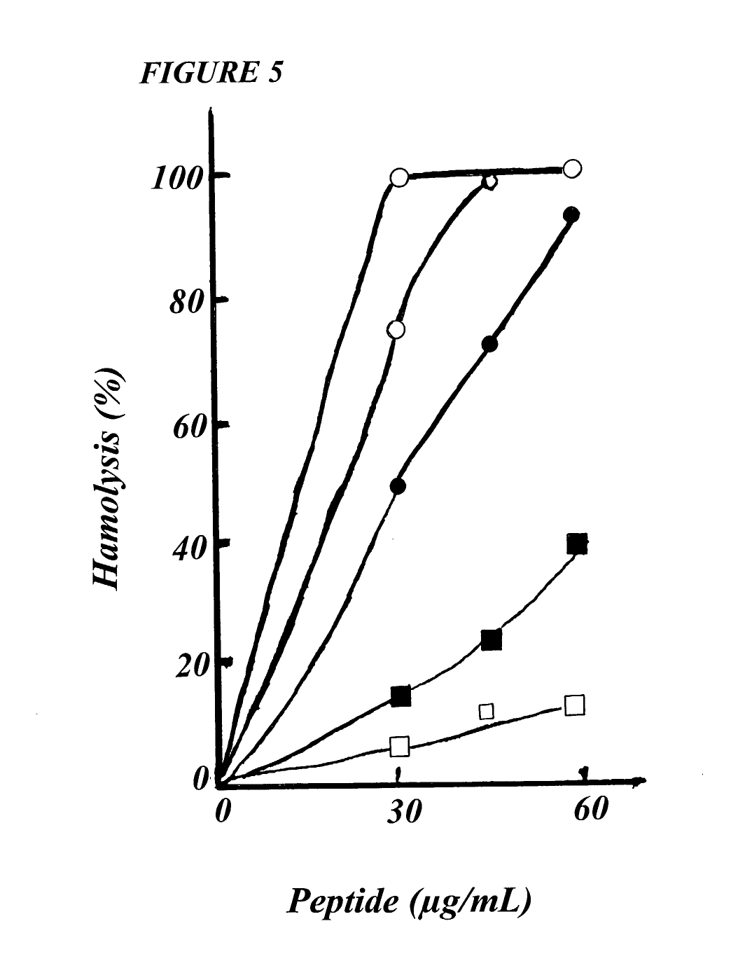

FIG. 5. Hemolysis of rat erythrocytes as a function of peptide concentration concentration Erythrocytes (−10⁶/ml) were incubated in Hepes buffered isotonic saline containing various concentrations of SPFK analogs. SPFK (A). Other symbols same as that in FIG. 1.

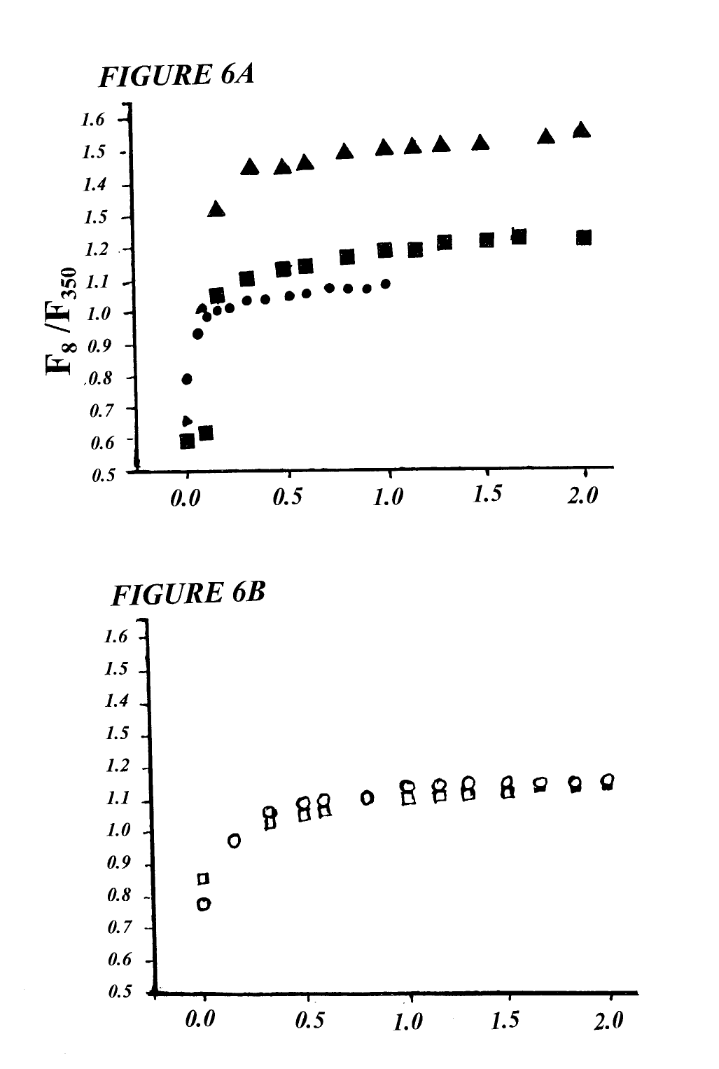

FIG. 6. Flourescence titration of SPFK and its analogs with calmodulin. SPFK or one of its analogs (3 μM) was titrated with calmodulin in 5 mM Tris-HCl buffer pH 7.4 containing 150 mM NaCl and 1 mM CaCl₂. The excitation wavelength was 295 nm. Emission spectra was recorded between 320 and 400 nm. Ratio of fluorescence at 335 (Fₐ) and 350 nm (F3₅₀) is plotted as a function of calmodulin equivalents. Panel (A) SPFK, A; Rsac, 0; Rsam. Panel (B) Dsac.D; Dsam,O

In another embodiment of the present invention, the helix is amphiphilic with well-defined polar and hydrophobic faces as is evident from the helical wheel diagram shown in FIG. 1. This is also reflected in its hydrophobic moment value of 0.42, which would fall in the surface-seeking region of the Eisenberg plot (29). The retro analogs of SPFK, RSac and RSam have the same amino acid composition as SPFK, but with a reversed sequence and hence their mean hydrophobicity (<H>) and hydrophobic moment (<μM>) values would be identical to that of SPFK, assuming that they would also adopt a-helical structure. However, in the case of diastereo isomers DSac and DSam, the <H> would be same as that of SPFK, but their <μH> would be greatly reduced, as their helical structure would be disrupted due to the presence of D-Leu residues at positions 4 and 8 of 13-residue peptides. Their exact <μH> values cannot, however be calculated as the structure they would assume would be unknown.

In further embodiment of the present invention, the retention times (RT) of the peptides on a reverse phase (C18)-HPLC column run under identical conditions are also summarized in Table 1. RSac and RSam have RT values 35.5′ and 36.62′ as compared to 32.1′ of SPFK, while the diastereomers DSac and DSam have RTs of 28.69′ and 28.79″ respectively. There appears to be a direct correlation between the RTs of peptides with same amino acid composition and their abilities to adopt amphiphilic structures (30, 31). The retention times indicate that RSac and RSam are capable of forming amphiphilic helical structures. DSac and DSam elute earlier as compared to SPFK and its retro isomers indicating that they 10 have lower propensity for helical structure due to the presence of D-Leu residues.

In further embodiment of the present invention, the helical propensities of the peptides were further examined by circular dichroism (CD) spectroscopy. The spectra of RSac and RSam in TFE shown in FIG. 2 are typical of a-helical conformation, with double minima at 222 and 208 nm and a cross over ˜200 nm. The spectra of DSac and DSam indicate low propensity for ordered structure even in TFE. The percentage helical contents of RSac and RSam calculated from their θ₂₂₂ values, were 30% and 35% respectively. All the peptides exhibited spectra characteristic of random conformation in aqueous medium.

In another embodiment of the present invention, the antimicrobial activities of the peptides were examined and the results are tabulated in Table 2 as shown at the end.

With the exception of DSac, all the other peptides inhibited the growth of the microorganisms tested, at comparable concentrations. Both the retro peptides RSac and RSam are almost as effective as SPFK on all the strains tested indicating that the sequence of side chains as oriented from the N to the C terminus is not important for the antimicrobial activity. Among the diastereomeric peptides, DSac is totally inactive, while DSam exhibits activity with MIC's comparable to that of SPFK and its retro isomers. While SPFK, RSac and RSam were also found to be completely bactericidal at their MICs, DSam was bactereostatic at 30 μg/l on E. coli and was bactericidal>50 μg/ml.

In yet another embodiment of the present invention, the mechanism by which cationic antimicrobial peptides kill bacteria involves permeabilization of the bacterial membrane (1, 11). Hence, we examined the ability of these peptides to permeabilize the bacterial outer membrane (OM) and cytoplasmic membrane (IM). The data presented FIG. 3 reveal that RSac is the most effective in permeabilizing the OM of E. coli. RSam and DSam are slightly less effective as compared to RSac. DSac does not permeabilize the OM effectively. The IM permeabilizing activity of the peptides are shown in FIG. 4. The activities of RSac and RSam are comparable whereas DSam is less effective in permeabilizing the IM. Peptide DSac is inactive.

In still another embodiment of the present invention, the antimicrobial activities of the retro analogs of SPFK arise from their abilities to permeabilize bacterial membranes. Peptide DSac is inactive, as it is unable to permeabilize the bacterial membranes. However, conversion of the C-terminal acid to amide results in the amidated peptide exhibiting antibacterial activity at concentrations which is only slightly lower than SPFK and the retero peptides. These results indicate that although DSam has very little propensity to adopt helical conformation positive charges are sufficient for antimicrobial activity.

In further embodiment of the present invention, the hemolytic activities of SPFK and the retro and diastereomeric analogs were determined and the data are shown in FIG. 5. SPFK exhibits 100% hemolysis at 30 μg/ml. Both the retro analogs of SPFK also exhibit hemolytic activity. Amongst the diastereomers, DSac shows very little hemolytic activity, whereas DSam shows −40% hemolysis at 60 μg/ml. At this concentration, SPFK and the retro peptides show 95-100% hemolysis. The loss of structure as a result of introducing D-amino acids results in selective decrease in its hemolytic activity.

In further embodiment of the present invention, cationic amphiphilic alpha helical segments of proteins and peptides have been recognized as calmodulin (CaM)-binding motifs (32). SPFK and its retro isomers have propensity for α-helical structure, while the diastereo isomers do not appear to fold into ordered structure. Hence, the ability of these peptides to interact with CaM was investigated. SPFK and its analogs contain a single Trp residue each and CaM is devoid of this amino acid. Therefore, the interaction of these peptides with CaM was studied by monitoring the change of Trp fluorescence. The results are summarized in Table 3 as shown at the end.

In another embodiment of the present invention, In buffer, SPFK has a λ max of 354 nm indicating that the Trp residue is exposed to aqueous environment. In the presence of CaM and 1 mM Ca²⁺ (molar ratios of 1:2 of CaM:SPFK) considerable blue shift in λ max (330 nm) and a >3 fold increase in fluorescence is observed. Since Ca²⁺ alone does not have any effect on the fluorescence spectra of SPFK, the observed fluorescence changes arise from the binding of peptide to CaM, resulting in the location of Trp in a hydrophobic environment. Other analogs of SPFK also show a λ max of −350 nm and upon addition of CaM in the presence of Ca²⁺, exhibit changes in fluorescence properties were very similar to that observed in the case of SPFK.

In yet another embodiment of the present invention, this indicates that all the analogs bind to CaM. However, the complex formed by the diastereo isomers were completely reversible upon addition of EDTA, whereas the complex formed by CaM with SPFK and its retro isomers were not completely disassociated in the presence of EDTA. Thus, in the absence of Ca²⁺ SPFK and RSam are capable of forming low-affinity complexes with CaM as indicated by less pronounced blue shift and smaller enhancement of Trp fluorescence. Such Ca²⁺-independent complexes are not formed by CaM with the diastereo analogs. The k_{d} value, 1.2×10′⁷ M, calculated from fluorescence-titration curves (FIGS. 6A and B) also indicate strong binding of SPFK to CaM. Retro analogs RSac and RSam have dissociation constants in the same range whereas the diastereo isomers have considerably higher k_{d} values. The loss of structure as compared to SPFK and the retro peptides in the diastereo analogs appears to have a direct bearing on its CaM-binding behavior.

In further embodiment of the present invention, applicants have synthesized diastereo isomers of SPFK. As one turn in a α-helix requires 4 amino acid residues, introduction of D-amino residues at the 4^{th} and 8^{th} positions in a 13-residue peptide like SPFK could be expected to effectively disrupt the structure.

In another embodiment of the present invention, the CD spectra of these analogs confirm the absence of a-helical structure in diastereo analogs and the short retention times on RP-HPLC show their inability to acquire any amphiphilic structure. However, DSac, the C-terminal free acid diastereo analog of SPFK, failed to exhibit any antimicrobial activity against any of the microorganisms tested which arises as a result of its inability to permeabilize both OM and IM of E. coli. Amidation at the C-terminal of DSac increases the net positive charge, which results in the peptide possessing antibacterial activity and hemolytic activity to a small extent. Introduction of 3 D-amino acid residues into magainin 2 has earlier been reported to result in total loss of its biological activity (50). Magainin 2 exhibits only antimicrobial activity and no hemolytic activity and this has been attributed to a delicate balance between hydrophobicity, hydrophobic moment and cationicity in this molecule (51). Changes in any one of these parameters result in total loss of biological activity. DSam, the amidated analog of DSac, exhibits antimicrobial activity with MICs comparable to that of SPFK and the retro analogs. Applicants have observed that even two D-amino acids are sufficient to disrupt ordered structure. In short peptides only the net charge on the peptide and hydrophobicity appears to be the critical factors for antibacterial activity even in the absence of ordered structure.

In yet another embodiment of the present invention, one of the additional advantages of introducing a few enantiomeric amino acid residues in the middle of the sequence of an amphiphilic peptide has been thought to be the reduction or abolition of other cytopathic effects of the peptide. Some of these cytopathic effects may arise from their ability to function as calmodulin antagonists through their binding to calmodulin, a major regulator of calcium dependent enzymes in eukaryotic cells (52). Hence the interaction of these peptides with calmodulin were investigated. Change in the fluorescence characteristics of Trp containing peptides in the presence of calmodulin has been used as a reliable indicator of their interaction with calmodulin (53). Our results demonstrate that SPFK and all the other analogs used n the study interact with calmodulin

In still another embodiment of the present invention, a qualitative difference as well as a reduced affinity in the binding of the diastereo isomers are discernible. It is conceivable that further reduction in its affinity can be brought about by introducing additional D-amino acid residues in the middle of the sequence without adversly affecting its beneficial effects like its antimicrobial activity.

In further embodiment of the present invention, our study indicates that reversing the sequence in a short amphiphilic peptide may not always result in selective loss of biological activity like hemolytic activity. Also, introduction of enantiomeric amino acids in a short peptide to generate a diastereomer could result in loss of structure as well as antimicrobial and hemolytic activities, unless compensated by increase in positive charges.

### REFERENCES

1. Saberwal, G. and Nagaraj, R. (1994) Cell-lytic and antibacterial peptides that act by perturbing the barrier function of membranes facets of their conformational features, structure-function correlations and membrane-perturbing abilities. Biochim. Biophys. Acta 1197, 109-131.

2. Lehrer, R., Lichenstein, A. K. and Ganz, T. (1993) Defensins Antimicrobial and cytotoxic peptides of mammalian cells, Annu. Rev. Immunol. 11, 105-128.

3. Nicolos, P. and Mor, A. (1995) Peptides as weapons against microorganisms in the chemical defense system of vertebrates. Annu. Rev. Microbiol. 49, 277-304.

4. Boman, H. G. (1995) Peptide antibiotics and their role in innate immunity. Annu. Rev. Immunol. 13, 61-92.

5. Maloy, W. L. and Kari, U. P. (1995) Structure-activity studies on magainins and other host defense peptides. Biopolymers 37, 105-122.

6. Hancock, R. E. W. and Chapple, D. S. (1999) Peptide antibiotics Antimicrob. Agents Chemother. 43, 1317-1323.

7. Ganz, T. and Lehrer, R. I. (1999) Antibiotic peptides from higher eukaryotes: Biology and applications. Mol. Med. To-day 5, 292-297.

8. Hancock, R. E. W. and Lehrer, R. (1998) Cationic peptides: A new source of antibiotics, Trends Biotech. 16, 82-88.

9. Hancock, R. E. W. (1997) Peptide antibiotics, Lancet 349, 418-422.

10. Nagaraj, R. (1997) Structure-function relationships and engineering of host-defense peptides. Current Sci. 72, 819-825.

11. Sitaran, N. and Nagaraj, R. (1999) Interaction of antimicrobial peptides with biological and model membranes: structural and charge requirements for activity. Biochim. Biophys. Acta 1462, 29-54.

12. Neu, H. C. (1992) The crisis in antibiotic resistance. Science 257, 1064-1073.

13. Silver, L. L. and Bostian, K. A. (1993) Discovery and development of new antibiotics: The problem of antibiotic resistance. Antimicrob. Agents Chemother. 37, 377-383.

14. Swartz, M. N. (1994) Hospital-acquired infections: Diseases with increasingly limited therapies. Proc. Natl. Acad. Sci. USA 91, 2420-2427.

15. Chopra, I., Hodgson, J. Metcalf, B. and Poste, G. (1997) The search for antimicrobial agents effective against bacteria resistant to multiple antibiotics, Antimicrob. Agents Chermother. 41, 497-503.

16. Austin, D. J., Kristinsson, K. G. and Anderson, R. M. (1999) The relationship between the volume of antimicrobial consumption in human communities and the frequences of resistance. Proc. Natl. Acad. Sci. USA 96, 1152-1156.

17. Hultmark D., Steiner, H., Rasmuson, T. and Boman, H. G. (1980) Insect immunity. Purification and property of three inducible bactericidal proteins from hemolymph of immunized pupae of Hyalophora cecropia. Eur. J. Biochem. 106, 7-16.

18. Bevins, C. L. and Zasloff, M. (1990) Peptides from frog skin. Annu. Rev. Biochem. 59, 395-410.

19. Shivali, S. Scheit, K. H. and Bhargava, P. M. (1989) seminalplasmin. In Proteins of Seminal plasma. pp 337-349. J. Wiley, NY.

20. Reddy, E. S. P. and Bhargava, P. M. (1979) Seminalplasmin—an antimicrobial protein from bovine seminal plasma which acts in E. coli by specific inhibition of rRNA synthesis. Nature 279, 725-728.

21. Sitaran, N, Krishnakumaral, V. and Bhargava, P. M. (1986) Seminalplasmin and caltrin are the same protein. FEBS Lett. 201, 233-236.

22. Sitaran, N. and Nagaraj, R. (1995) Seminalplasmin. Bio Essays 17, 415-422.

23. Sitaran, N. and Nagaraj, R. (1990) A synthetic 13-residue peptide corresponding to the hydrophobic region of bovine seminalplasmin has antibacterial activity and also causes lysis of red blood cells, J. Biol. Chem. 265, 10438-10442.

24. Sitaran, N. Chandy, M. Pillai, V. N. R. and Nagaraj, R. (1992) Change of glutamic acid to lysine in a 13-residue antibacterial and hemolytic peptide results in enhanced antibacterial activity without increase in hemolytic activity. Antimicrob. Agents Chemother. 36, 2468-2471.

25. Atherton, E. and Sheppard, R. C. (1989) Solid Phase Peptide Synthesis: A practical Approach. IRL, Oxford.

26. Loh, B. Grant, C. and Hancock, R. E. W. (1984) Use of fluorescent probe 1-N-phenylnaphthylamine to study the interactions of aminoglycoside antibiotics with outer membrane of Pseudomonas aerugenosa. Antimicrob. agents Chemother. 26, 546-551.

27. Miller, J. H. (1972) Experiments in Molecular Genetics. Cold Spring Harbor, NY.

28. Wu, C. S., Ikeda, K. and Yang, J. T. (1981) Ordered conformation of polypeptides and proteins in acidic dodecyl sulfate solution. Biochemistry, 20, 566-570.

29. Eisenberg, D., Schwarz, E., Komarony, M. and Wall, R. (1984) Analysis of membrane and surface protein sequences with hydrophobic moment plot. J. Mol. Biol. 179, 125-142.

30. Blondelle S. E. and Houghten R. A. (1991) Hemolytic and antimicrobial activities of the twenty-four individual omission analogues of melittin. Biochemistry 30, 4671-4678.

31. Krause, E. Beyermann, M., Dathe, M., Rothermund, S. and Bienert, M. (1995) Location of an amphipathic α-helix in peptides using reversed-phase HPLC retention behavior of D-amino acid analogs. Anal. Chem. 67, 252-258.

32. O'Neil, K. T. and DeGrado, W. F. (1990) How calmodulin binds its targets: Sequence independent recognition of amphiphilic α-helices. Trends Biochem. Sci. 15, 51-64.

33. Fennell, J. F., Shipman, W. H. and Cole, L. J. (1968) Antibacterial action of melittin, a polypeptide from bee venom. Proc. Soc. Exp. Biol. Med. 127, 707-710.

34. Dempsey, C. (1990) The actions of melittin on membranes. Biochim. Biophys. Acta 1031, 143-161.

35. Shai, Y. (1994) Pardaxin: Channel formation by a shark repellant peptide from fish. Toxicology 87, 109-129.

36. Dhople, V. M. and Nagaraj, R. (1993) δ-toxin unlike melittin has only hemolytic activity and no antimicrobial activity: rationalization of this specific biological activity. Biosci. Rep. 13, 245-250.

37. Javvadi, R. Vunnam, S. and Merrifield, R. B. (1996) Synthetic melittin, its enantic, retro and retroenantic isomers and selected chimeric analogs: Their antibacterial, hemolytic and lipid bilayer action. J. Am. Chem. Soc. 118, 8989-8997.

38. Blondelle, S. E. and Houghten, R. A. (1991) Probing the relationships between the structure and hemolytic activity of melittin with a complete set of leucine substitution analogs. Pept. Res. 4, 12-18.

39. Sitaran, N., Subbalakshmi, C. and Nagaraj, R. (1995) Structural and charge requirements for antimicrobial and hemolytic activity in the peptide PKLLETFLSKWIG corresponding to the hydrophobic region of the antimicrobial protein bovine seminalplasmin. Int. J. Pept. Prot. Res. 46, 166-173.

40. Shal, Y., Bach, D. and Yamovsky, A. (1990) Channel forming property of synthetic pardaxin and analogues, J. Biol. Chem. 265, 20202-20209.

41. Pouny, Y. and Shai. Y. (1992) Interaction of antimicrobial dermaseptin and its fluorescently labeled analogues with phospholipid membranes. Biochemistry 31, 12416-12423.

42. Perez-Paya, E., Houghten, R. A. and Blondelle, G. E. (1995) The role of amphipathicity in the folding, self-association and biological activity of multiple subunit small proteins. J. Biol. Chem. 270 1048-1056.

43. Thannarasu, S. and Nagaraj, R. (1995) Design of 16-residue peptides possessing antimicrobial and hemolytic activities or only antimicrobial activity from an inactive peptide. Int. J. Prot. Peptide Res. 46, 480-486.

44. Shai, Y. and Oren, Z. (1996) Diastereomers of cytolysins, a novel class of potent antibacterial peptides. J. Biol. Chem. 271, 7305-7308.

45. Oren, Z. and Shai, Y. (1996) A class of highly potent antibacterial peptides derived from pardaxin, a pore-forming peptide isolated from Moses sol fish Pardachinus marmoratus. Eur. J. Biochem. 237, 303-310.

46. Oren, Z. and Shai, Y. (1997) Selective lysis of bacteria but not mammalian cells by diastereomers of melittin: Structure-function study. Biochemistry 36, 1826-1835.

47. Oren, Z. Hong, J. and Shai, Y. (1999) A comparative study on the structure and function of a cytolytic α-helical peptide and its antimicrobial β-sheet diastereomer. Eur. J. Biochem. 259, 360-369.

48. Feder, R., Dagan, A. and Mor, A. (2000) Structure-activity relationship study of antimicrobial dermaseptin S4 showing the consequences of peptide oligomerization on selective cytotoxicity. J. Biol. Chem. 275, 4230-4238.

49. Oren, Z., Hong, J. and Shai, Y. (1997) A repertoire of novel antibacterial diastereomeric peptides with selective cytolytic activity. J. Biol. Chem. 272, 14643-14649.

50. Chen, H. Brown, J. H. Morell, J. L. and Huang, C. M. (1988) Synthetic magainin analogues with improved antimicrobial activity. FEBS Lett. 236, 462-466.

51. Matsuzaki, K. (1998) Magainins as paradigm for the mode of action of pore forming polypeptides. Biochim. Biophys. Acta 1376, 391-400.

52. Means, A. R., Putlay, J. A. and Epstein, P. (1988) in “Calmodulin” (Ed Cohen, P. and Klec, C. B. Elsevier pp. 17-33.

53. Erickson Vitamen, S. and DeGrado, W. F. (1987) Recognition and characterization of calmodulin-binding sequences in peptides and proteins. Method. Enzymol. 139, 455-478.

1. A 13-residue diastereo analog Dsam of SEQ ID No. 1 of peptide SPFK-amide having amino acid D-leucine at positions 4 and 8.

2. An analog as claimed in claim 1, wherein said analog shows hydrophobicity (H) value raging between 0.19-0.21.

3. An analog as claimed in claim 1, wherein said analog shows hydrophobicity moment of about 0.043.

4. An analog as claimed in claim 1, wherein said analog shows net charge of about +4.

5. An analog as claimed in claim 1, wherein said analog shows retention time of about 36.62 minutes on Reverse Phase High-Performance Liquid Chromatography.

6. An analog as claimed in claim 1, wherein said analog forms unordered structure in both aqueous and trifluoroethanol mediums.

7. An analog as claimed in claim 1, wherein said analog shows lower propensity for helical structure due to presence of D-Leu residues.

8. An analog as claimed in claim 1, wherein said analog shows anti-microbial activity.

9. An analog as claimed in claim 1, wherein said analog shows reduced hemolytic activity as compared to SPFK.

10. A method of producing a 13-residue diastereo analog Dsam of SEQ ID No. 1 of peptide SPFK-amide having amino acid D-leucine at positions 4 and 8 wherein said method comprises steps of:
(a) synthesizing said peptide by solid phase peptide synthesis method,
(b) purifying the said synthesized deprotected peptide by chromatography,
(c) confirming the purity of peptide by mass spectrometery.

11. A method as claimed in claim 10, wherein said peptide is purified by High pressure Liquid Chromatography.

12. A method as claimed in claim 10, wherein said peptide shows about same antimicrobial activity as SPFK.

13. A method as claimed in claim 10, wherein said peptide shows reduced hemolytic activity as compared to SPFK.

14. A method as claimed in claim 10, wherein said peptide shows anti-microbial activity by permeabilization of microbial membrane.

15. A method as claimed in claim 10, wherein said analog shows anti-microbial activity at concentration 50 μg/ml and above.

16. A method as claimed in claim 10, wherein said analog is administered through oral, nasal, intravenous, and/or intradermal route.

17. A composition useful for anti-microbial activity, said composition comprising diastereo analog Dsam of claim 1 and optionally pharmaceutically acceptable additives.

18. A composition as claimed in claim 17, wherein said analog and additives are in the ratio ranging between 1:1 to 1:10.

19. A composition as claimed in claim 17, wherein said additives are selected from a group comprising cellulose, magnesium stearate, calcium carbonate, starch-gelatin paste, and/or pharmaceutically acceptable carriers, excipient, diluent, or solvent.

20. A composition as claimed in claim 17, wherein the additives show no adverse effect on the activity of the analog.

21. A composition as claimed in claim 17, wherein said analog shows anti-microbial activity at concentration 50 μg/ml and above.

22. A composition as claimed in claim 17, wherein said composition shows reduced hemolytic activity as compared to SPFK.

23. A composition as claimed in claim 17, wherein said composition is administered through oral, nasal, intravenous, and/or intradermal route.
TABLE 1
Sequences of SPFK analogs and their physico-chemical properties

| | | | | Net Charge at | |
|---|---|---|---|---|---|
| Peptide | Sequence | <H> | <HH>> | pH 7.0 | Retention time(RT) on RPLC (min) |
| SPFK | PKLLKTFLSKWIG-COOH | 0.18 | 0.42 | +3 | 32.10 |
| Retro SPFK (RSac) | GIWKSLFTKLLKG-COOH | 0.21 | 0.43 | +3 | 35.5 |
| Retro SPFK | GIWKSLFTKLLKG-CONH₂ | 0.21 | 0.43 | +4 | 36.62 |
| amide (RSam) | | | | | |
| Diastereo SPFK | PKLLKTFLSKWIG-COOH | 0.18 | | +3 | 28.69 |
| (DSac)^{a} | | | | | |
| Diastereo SPFK | PKLLETFLSKWIG-CONH₂ | 0.18 | * | +4 | 28.79 |
| amide (DSam) | | | | | |

Bold letters correspond to amino acids in D-configuration
TABLE 2
Antimicrobial activity of SPFK and its analogs

| | Microorganisms | | |
|---|---|---|---|
| | | S. aureus | |
| Peptide | E. coli W 160.37 | MIC(μg/mL) | P. aeruginosa |
| SPFK | 7 | 5 | 30 |
| RSac | 5 | >5 | 25 |
| RSam | 5 | 10 | 15 |
| DSac | Inactive | Inactive | Inactive |
| DSam | 10 | 15 | 25 |

TABLE 3
Fluorescence properties of tryptophan in SPFK and analogs in buffer
and in the presence of calmodulin

| | λₘₐₓ (Fluorescence Intensity in arbitrary units) | | |
|---|---|---|---|
| | calmodulin | | |
| Peptide³ | Buffer | ImMCaCl₂ | 2mM EDTA |
| SPFK | 354 (24) | 330 (90) | 345 (33) |
| RSac | 350 (71.5) | 336 (113) | 349 (72) |
| RSam | 354 (29) | 329 (87) | 342 (36) |
| DSac | 352 (53) | 338 (134) | 348 (56) |
| DSam | 351 (53) | 334 (157) | 349 (57) |

Peptide concentration 3 μM.
EDTA was added after the stabilization of fluorescence in the presence of 1 mM Ca Cl₂.
